SEQUENCE LISTING (5 sequences)

SEQ ID NO: 1 - DNA, 5446 bp, artificial sequence
  pcDNA3 vector nucleotide sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 23 bp, artificial sequence
  Oligonucleotide CpG
tccataacgttgcaaacgttctg

SEQ ID NO: 3 - DNA, 23 bp, artificial sequence
  Oligonucleotide GpC
tccataagcttgcaaagcttctg

SEQ ID NO: 4 - protein, 24 aa, artificial sequence
  Hsp60 peptide analog p277
VLGGGVALLRVIPALDSLTPANED

SEQ ID NO: 5 - protein, 20 aa, artificial sequence
  Hsp60 peptide analog p12
EEIAQVATISANGDKDIGNI

(19) United States
(12) Patent Application Publication
(10) Pub. No.: US 2004/0005588 A1
(43) Pub. Date: Jan. 8, 2004

(54) Methods of treatment or prevention of autoimmune diseases with CpG-containing polynucleotide

(76) Inventors: Irun R. Cohen, Rehovot (IL); Francisco J. Quintana, Capital Federal (AR)

Correspondence Address: WINSTON & STRAWN, PATENT DEPARTMENT, 1400 L STREET, N.W., WASHINGTON, DC, 20005-3502, US

(21) Appl. No.: 10/371,116

(22) Filed: Feb. 24, 2003

Related U.S. Application Data
(63) Continuation of application No. PCT/IL01/00790, filed on Aug. 23, 2001.

(51) Int. Cl. C12Q 1/68

(52) U.S. Cl. 435/6

(57) ABSTRACT

The present invention relates to DNA vaccines useful for the prevention and treatment of ongoing autoimmune diseases. The compositions and methods of the invention feature the CpG oligonucleotide, preferably in a motif flanked by two 5′ purines and two 3′ pyrimidines. The vaccine may include DNA encoding a specific antigen, or the peptide antigen itself. The invention is exemplified for insulin-dependent diabetes mellitus.

### CROSS-REFERENCE TO RELATED APPLICATIONS

This application is a continuation of the US national phase designation of International application PCT/IL01/00790 filed Aug. 25, 2001, the content of which is expressly incorporated herein by reference thereto. This application also claims the benefit of provisional application 60/227,853 filed Aug. 25, 2000

### FIELD OF THE INVENTION

The present invention relates to methods for the prevention or treatment of autoimmune diseases and particularly insulin-dependent diabetes mellitus (IDDM), and more particularly to such methods in which the vaccine includes a DNA molecule which includes a CpG motif.

### BACKGROUND OF THE INVENTION

DNA vaccination is an efficient approach to induce protection against infectious pathogens (Tascon et al, 1996) and cancer (Stevenson et al, 1999), and to modulate autoimmune processes (Waisman et al, 1996). It has been shown that after intramuscular injection of a naked expression vector, plasmid DNA is taken up by muscle cells and maintained episomally, allowing the expression of the encoded antigen (Wolff et al, 1992). Thus after single or repeated injections of DNA, cellular and/or humoral immune responses to the encoded protein are mounted, and long-lived memory lymphocytes are induced (Hassett et al, 2000). These memory cells may have regulatory functions and, therefore, might serve as tools for the modulation of autoimmune conditions.

The CpG oligodeoxynucleotide (CpG-ODN) is an immunostimulatory sequence present primarily in bacteria (Lipford et al 1998; Krieg et al 1998; and Krieg et al, 1999).

Bacterial DNA contains immunostimulatory motifs consisting of a centralized unmethylated CpG dinucleotide flanked by two 5′ purines and two 3′ pyrimidines (Klinman et al, 1997). CpG motifs are underrepresented in mammalian genomes, due to a combination of CpG suppression and CpG methylation (Klinman et al, 1996). It has been reported that this motif stimulates Th1 responses in vivo (Klinman et al 1996). For this reason, it has been suggested that single stranded DNA containing this motif would serve as a powerful adjuvant for both humoral and cellular immune responses (Lipford et al 1997, Krieg et al 1998). It has also been reported that intratumoral injections of phosphorothioate oligodeoxynucleotides with CpG motifs induce rejection of established tumors and thus represent a new immunotherapeutic approach in human gliomas, which overcomes the need for the selection and purification of a tumoral antigen (Carpentier et al 2000).

The NOD mouse spontaneously develops insulin dependent diabetes mellitus (IDDM) as a consequence of an autoimmune process that leads to destruction of the insulin-producing β cells of the pancreas (Tisch et al, 1996). Several antigens have been identified as targets for diabetogenic T cells, including β-cell specific proteins such as insulin, non-β-cell restricted antigens such as GAD, and even ubiquitous antigens such as heat shock protein 60 (Hsp60, Tisch et al, 1996). It has been shown that the onset of diabetes is preceded by an increase in T cell reactivity towards Hsp60 and to an Hsp60 peptide comprising amino acids 437 and 460 of the intact molecule, named p277 (Elias et al, 1991). In contrast to the early T-cell reactivity, antibodies to Hsp60 and p277 can only be detected late in the natural history of the disease, months after the onset of clinical diabetes, when the destructive process has terminated (Krause et al, 1999). Peptide p277 administered to NOD mice in incomplete Freund's adjuvant can arrest the development of diabetes (Elias et al, 1995). Furthermore, p277 treatment is able to induce remission of advanced insulitis even after the clinical onset of hyperglycemia (Elias et al, 1994). Successful treatment is associated with down-regulation of spontaneous T-cell reactivity to p277 and with the induction of antibodies to p277; these antibodies have Th2 associated isotypes (IgG1 and IgG2b), otherwise not found in young NOD mice (Elias et al, 1997; Ablamunits et al, 1998).

The effect of bacterial DNA on autoimmune inflammation is known. Bacterial DNA contains immunostimulatory motifs consisting of a central unmethylated CpG dinucleotide flanked by two 5′ purines and two 3′ pyrimidines (Klinman et al, 1997). CpG motifs are under-represented in mammalian genomes, due to a combination of CpG suppression and CpG methylation (Klinman et al, 1996). However, modulation of autoimmune conditions by bacterial DNA has been already reported. Gilkeson et al (1996), demonstrated that immunization with bacterial DNA can modulate renal disease in autoimmune NZB/NZW mice, while calf thymus DNA was not effective (Gilkeson, 1996). Furthermore, improvement in renal disease was associated with the induction of antibodies to glomerular antigens immediately after immunization (Gilkeson et al, 1996). Boccacio and her colleagues have reported that non-coding plasmid DNA can inhibit EAE attributed to its ability for activating IFN_{γ} in vivo (Boccaccio et al, 1999).

Nowhere in the prior art it is taught or suggested that DNA molecules which includes a CpG motif may be used as a vaccine for prevention or treatment of an naturally ongoing or spontaneous autoimmune disease in general or IDDM in particular.

### SUMMARY OF THE INVENTION

It is an object of the present application to provide vaccines comprising a DNA molecule which includes a CpG motif. It is another object of the invention to provide methods for the prevention or treatment of autoimmune diseases, particularly insulin-dependent diabetes mellitus (IDDM).

In order to explore the potential of a DNA-based therapy of diabetes, the present inventors set out to investigate whether immunization with a DNA construct encoding the heat shock protein-60 (Hsp60) could modulate autoimmunity and prevent the onset of the disease. Surprisingly, not only the Hsp60 containing construct, but also the empty vector (pcDNA3) were capable of reducing the incidence of diabetes. Indeed, the CpG oligonucleotide motif present in the construct could, by itself, be used to inhibit the development of NOD diabetes. Despite the absence of Hsp60, effective treatment was associated with specific immune effects on Hsp60 autoreactivity: down-regulation of the spontaneous T-cell proliferation to Hsp60 and to its peptide analog p277(Val⁶-Val¹¹) and the induction of specific antibodies to these molecules.

Accordingly, the present invention relates to a method for the treatment or prevention of autoimmune diseases by administering a DNA vaccine which is a molecule which includes a CpG motif. The CpG motif is preferably the dinucleotide CG flanked on the 5′ side by two purines and on the 3′ side by two pyrimidines and is most preferably AACGGT.

The present invention further relates to DNA vaccines comprising DNA sequences encoding a peptide or polypeptide antigen associated with autoimmune diseases, particularly IDDM.

These vaccines, may further include DNA encoding an antigen which has previously been used for the treatment of diabetes including Hsp60, p277, p277(Val⁶-Val¹¹) and p12 as well as any other such antigen disclosed, for example, in U.S. Pat. Nos. 5,780,034, 6,096,314, 6,180,103 and 6,110,746 and in international publications WO96/19236 and WO97/01959 (the entire contents of each of which being hereby incorporated herein by reference), rather than DNA encoding such antigens, the vaccine may also include the peptide or polypeptide antigens themselves. These peptide or polypeptide antigens may be administered simultaneously with or independent from the DNA vaccine. Methods for prevention and treatment autoimmune diseases comprising administering such DNA vaccines alone or together with such DNA or peptide molecules are within the scope of the present invention.

Use of a DNA molecule comprising a CpG motif for the preparation of a vaccine for treatment or prevention of an ongoing autoimmune disease, especially IDDM, represent another aspect of the invention. In particular, the invention relates to an improvement in a method of preparing a vaccine for treatment or prevention of an ongoing autoimmune disease, wherein the improvement comprises incorporating in the vaccine a DNA molecule comprising a CpG motif.

### BRIEF DESCRIPTION OF THE DRAWINGS

FIGS. 1A and 1B are graphs showing antibodies to Hsp60 in BALB/c mice immunized with the plasmid pHsp60.

FIG. 2 is a graph showing prevention of NOD diabetes by DNA vaccination.

FIG. 3 is a graph showing reduction of insulitis by DNA vaccination.

FIGS. 4A and 4B are graphs showing proliferative responses to Hsp60 and p277(Val⁶-Val¹¹) in DNA-vaccinated mice.

FIGS. 5A and 5B are graphs showing induction of antibodies to Hsp60 and p277(Val⁶-Val¹¹) by vaccination with plasmids or the CpG oligonucleotide.

FIG. 6 is a graph showing prevention of NOD diabetes by CpG injection.

FIGS. 7A and 7B are graphs showing isotypes of antibodies to Hsp60 and p277(Val⁶-Val¹¹) induced by vaccination with plasmids or the CpG oligonucleotide.

FIGS. 8A and 8B are graphs showing production of IL-10 and IFN_{γ} in response to the CpG oligonucleotide in NOD spleen cell cultures.

FIG. 9 is a graph showing that activation of splenocytes with CpG leads to Hsp60 release.

### DETAILED DESCRIPTION OF THE INVENTION

It is an object of the present application to provide vaccines comprising a DNA molecule which include a CpG motif. It is another object of the invention to provide methods for the prevention or treatment of autoimmune diseases, particularly insulin-dependent diabetes mellitus (IDDM).

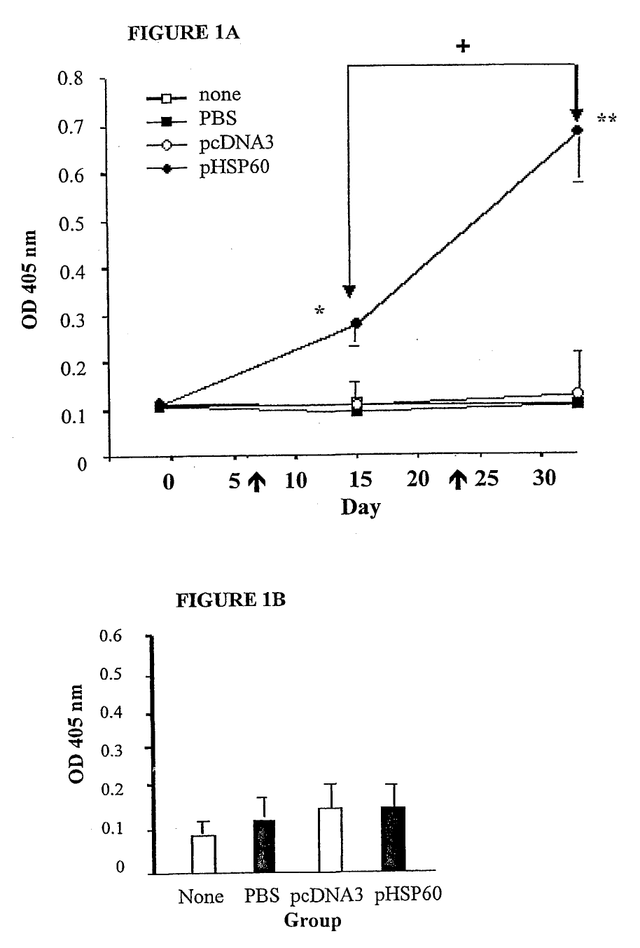

The present invention was discovered in the course of an investigation to test the effectiveness of DNA vaccination with Hsp60 as a specific immunotherapy for NOD diabetes. The specific immunogenicity of the pHsp60 plasmid in BALB/c mice (FIGS. 1A and 1B) was first ascertained. However, three unexpected observations were made when DNA treatment was used in NOD mice.

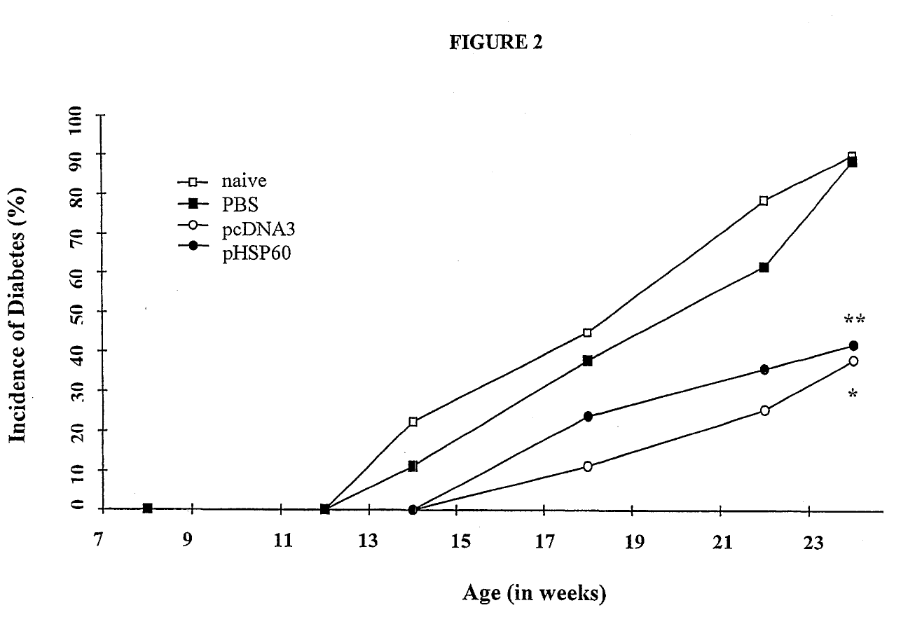

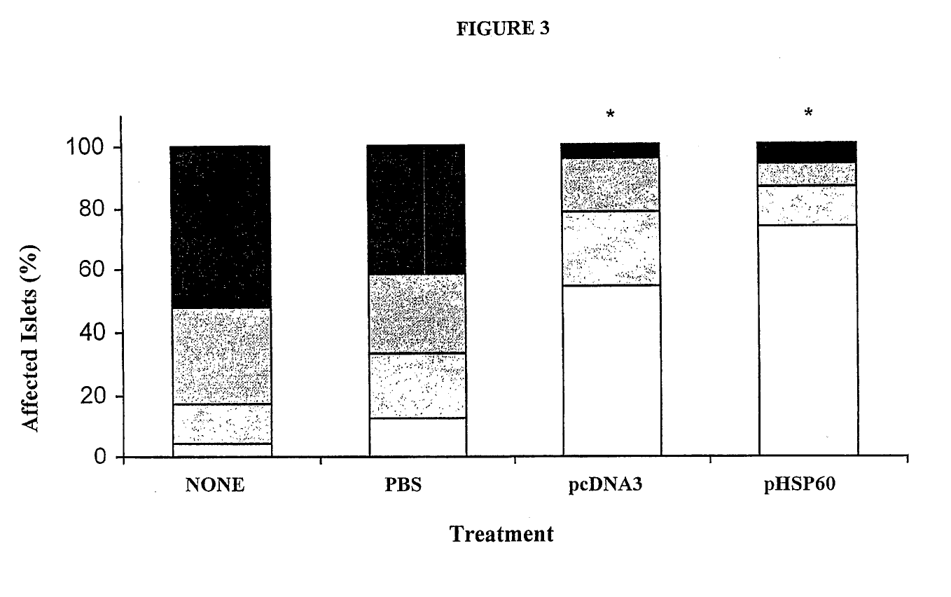

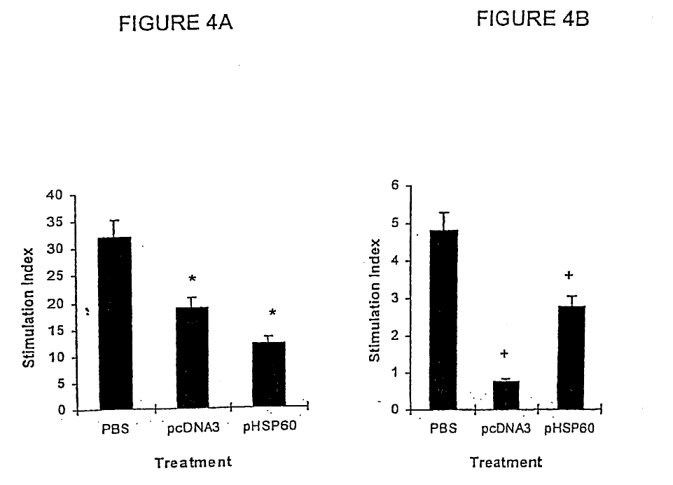

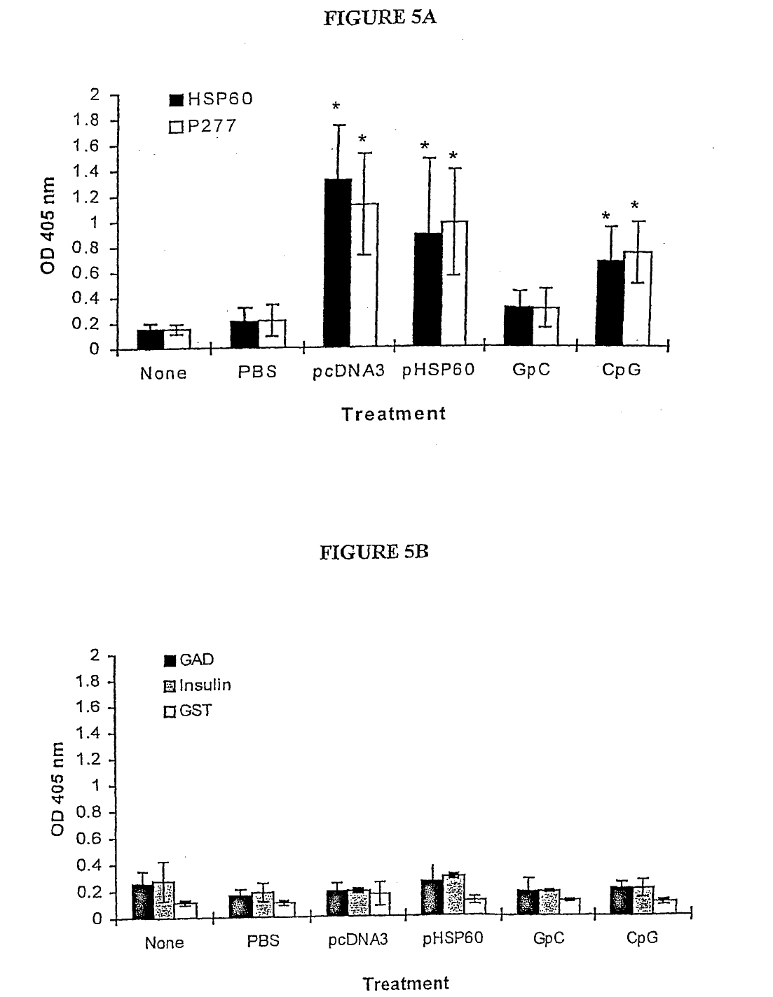

First, the pcDNA3 plasmid, which did not contain any sequences encoding Hsp60, was as effective in inhibiting the development of diabetes as was the pHsp60 plasmid (FIGS. 2 and 3). Secondly, the pcDNA3 plasmid, despite the absence of Hsp60, could still induce specific effects on the autoimmunity to Hsp60 intrinsic to the NOD diabetogenic process: down-regulation of T-cell proliferation and the induction of IgG2b antibodies to whole Hsp60 and to its peptide analog p277(Val⁶-Val¹¹). Responses to other antigens implicated in NOD diabetes, GAD and insulin, were not detected (FIGS. 4A, 4B, 5A, 5B, 7A and 7B). Thirdly, the CpG oligonucleotide by itself could essentially reproduce the effects of the pcDNA3 plasmid on Hsp60 autoimmunity and on the diabetes (FIGS. 5A, 5B, 6, 7A and 7B).

The CpG oligonucleotide is an immunostimulatory sequence present primarily in bacteria (Lipford et al, 1998; Krieg et al, 1998; and Krieg, 1999), and the present results using CpG might explain one of the mechanisms by which bacterial infections can inhibit the development of diabetes in NOD mice (Atkinson et al, 1999); bacterial infections may supply CpG stimulation.

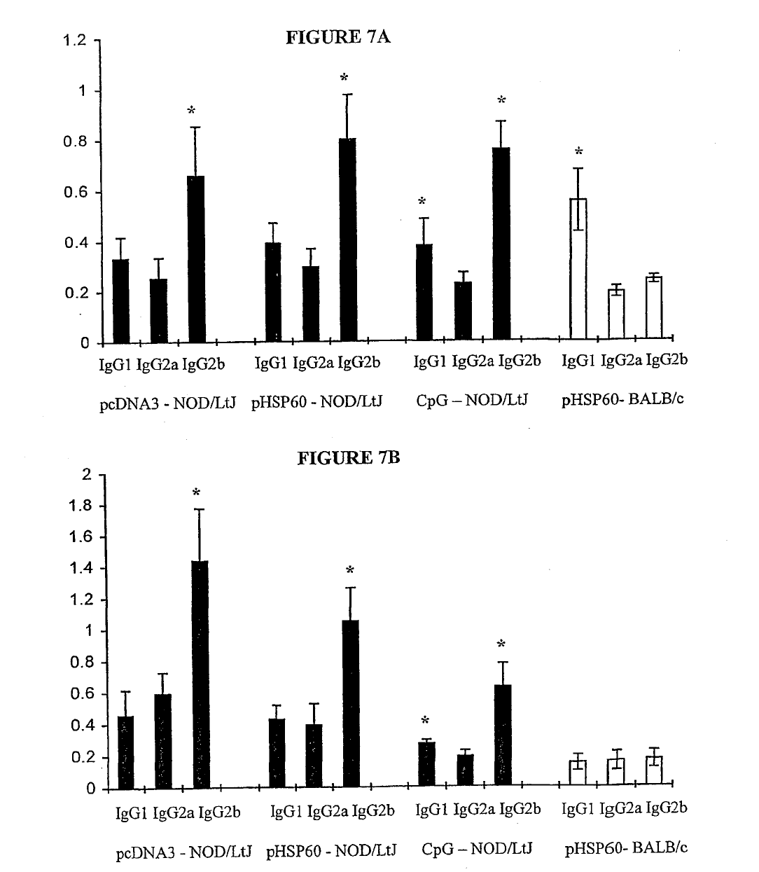

It is noteworthy that the antibodies to Hsp60 and to peptide p277(Val⁶-Val¹¹) were of the IgG2b isotype (FIGS. 7A and 7B). The cytokine required for the production of IgG2b antibodies is TGFα, known for its suppressive effects (McIntyre et al, 1993 and Snapper et al, 1993). TGFα is a Th2-associated cytokine, which has been shown to protect NOD mice from diabetes (King et al, 1998). Although DNA vaccination also induced Hsp60 and p277(Val⁶-Val¹¹) specific antibodies of the IgG2a subclass, considered to be IFN_{γ} dependent, the amount of these antibodies was significantly less than the amount of IgG2b antibodies. Thus, the cytokine balance was weighted more towards a Th2 response, suggesting that the therapeutic effects of DNA might be related to the activation of Th2-like T cells. Activation of Th2-like T cells was also described when spontaneous diabetes of NOD was prevented by the administration of the Hsp60 derived peptides p12 or p277 (Elias et al, 1997 and Bockova et al, 1997). Such T cells might suppress the Th1 T cells thought to be involved in the damage to the β-cells (Bockova et al, 1997).

The origin of the antibodies to Hsp60 and p277(Val⁶-Val¹¹) in mice protected from diabetes by pcDNA3 or treatment with the CpG oligonucleotide, or induced by the CpG oligonucleotide (FIG. 5A) is strain specific. BALB/c mice did not produce these antibodies when injected with pcDNA3 (FIG. 1A). NOD mice seem to manifest a spontaneous autoimmune response to Hsp60 and p277(Val⁶-Val¹¹), which is depicted in FIGS. 4A and 4B. Immunity to Hsp60 and p277 manifests as a peak of T cell reactivity before the onset of the disease (Elias et al, 1999 and Birk et al, 1996). Months after the onset of overt diabetes, antibodies to Hsp60 and p277 can be detected (Krause et al, 1999). After DNA treatment, the T cell proliferative response was diminished and replaced by the production of antibodies, mostly IgG2b. This suggests that the pre-existing autoimmune response, spontaneously arising in NOD mice, changes its phenotype after activation by bacterial DNA or CpG motifs, leading to the induction of Th2-like, IgG2b antibodies. Similarly, prevention of NOD diabetes by idiotypic induction of lupus with a monoclonal antibody was also associated with the induction of specific antibodies to Hsp60 and p277 (Krause et al, 1999). Thus, even when the induction of antibodies to Hsp60 and to p277 does not result from specific immunization, the appearance of such antibodies seems to serve as an indicator of the arrest of the diabetogenic process.

The CpG motif stimulates Th1 responses in vivo (Klinman et al, 1996). Unexpectedly, this motif classically associated with a Th1 phenotype is now disclosed to be effective in inhibition of diabetes, known to be Th2 mediated. This paradox has been observed in animal models of spontaneous diabetes. Poly-I:C, IFN_{γ}, IL-12, TNFα and IL-18, all of them well known inducers or mediators of Th1 responses, were shown to decrease insulitis and prevent diabetes (Campbell et al, 1991; Nicoletti et al, 1998; Rothe et al, 1999; Serreze et al, 1989; Sobel et al, 1998; and Yang et al, 1994). Furthermore, in the case of IL-18, protection was associated with systemic activation of Th1 type immunity, together with a shift to a Th2 phenotype of the cells infiltrating the islets (Rothe et al, 1999). Therefore, non-specific stimulation of the NOD immune system, even by Th1 inducers, is able to reset the ongoing immune response to islet antigens and arrest the diabetogenic process.

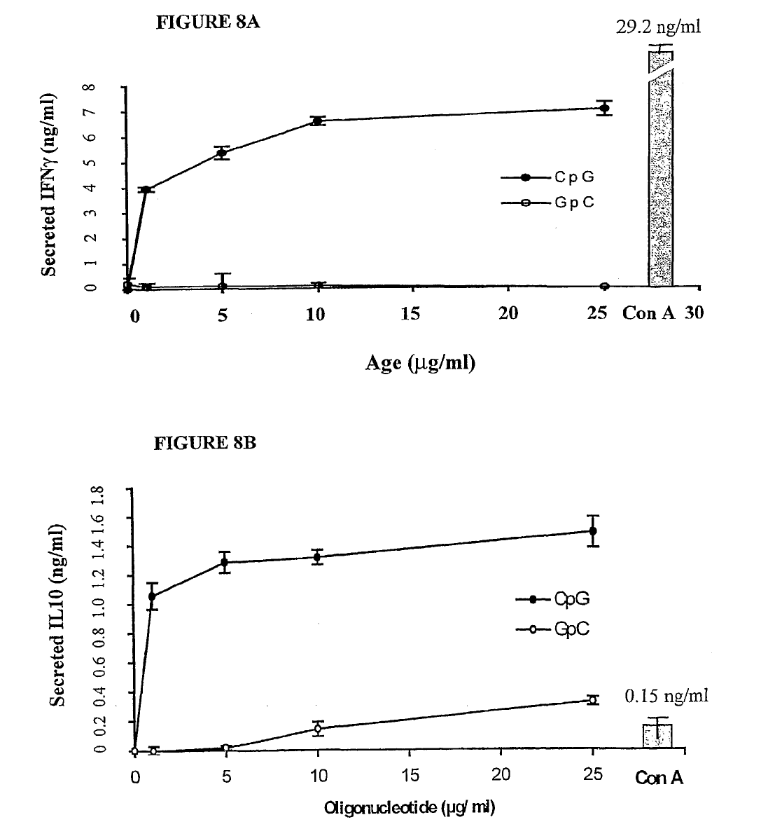

When the effect of the CpG oligonucleotide on NOD spleen cells was analyzed in vitro, it clearly induced IFN_{γ} and IL-10, in a dose dependent manner (FIGS. 8A and 8B). However, when the amounts of cytokine produced by CpG were compared to those triggered by Con A stimulation, it was evident that the effect of the CpG motif favored on the release of IL-10. Perhaps the prominence of IL-10 is important in modulating the diabetogenic process.

It was further found that the effect of CpG on the natural course of spontaneous NOD-diabetes appears to involve two mechanisms: a. a non-specific effect on APC function. b. a specific effect on the T-cell response to diabetes associated antigens. As demonstrated herein, CpG stimulation of splenocytes leads to the upregulation and secretion of Hsp60, and the activation of Hsp60-specific T-cells. Furthermore, in comparison to activation of Hsp60-specific T-cells through the addition of exogenous peptide, CpG stimulation shifts the phenotype of activated T-cells towards Th2. Since Hsp60 T-cells directed to the p12 and p277(Val⁶-Val¹¹) epitopes have been shown to regulate the progression of spontaneous NOD-diabetes, the effect of CpG on activated anti-p277(Val⁶-Val¹¹) and anti-p12 Th2 immunity might explain its modulatory effect on diabetes.

In addition, CpG affects APC function in NOD mice, probably through an IL-10 mediated mechanism. This change in APC function leads to downregulation and shift of the self-reactivity directed to diabetes-associated antigens from the pathogenic Th1 phenotype to a protective Th2 response.

Without being restricted to any particular mechanism, it is speculated that, prevention of NOD-spontaneous diabetes by CpG involves the specific regulation of Hsp60T-cell mediated autoreactivity, as well as changes in APC function that led to spreading of the Th2 shift to other diabetes-associated antigens.

Accordingly, the present invention is directed to a method for the prevention of all autoimmune diseases and particularly for the prevention of insulin-dependent diabetes mellitus (IDDM). The method involves vaccinating individuals with an effective amount of a DNA vaccine which includes a CpG motif. This same method of vaccination can be used for the treatment of autoimmune diseases and particularly for the treatment of IDDM.

The oligonucleotide with a CpG motif is preferably one which includes the dinucleotide CG flanked on the 5′ side by two purines and on the 3′ side by two pyrimidines. The nucleotides A and G are purines and the nucleotides C and T are pyrimidines. The precise purines and pyrimidines can vary, although the motif is preferably AACGTT. This six nucleotide motif is the smallest size that can be used for the vaccine, but the total length of the construct used for the vaccine is unlimited as is evidenced by the efficacy of the pcDNA3 empty vector which contains this motif. Those of ordinary skill in the art will be aware of oligonucleotides containing the CpG motif which have been used in the literature for various experimentation and any of these oligonucleotides can be used for the purpose of the present invention. The oligonucleotide of SEQ ID NO:2 is only one non-limiting example of such an oligonucleotide. It will be noted that SEQ ID NO:2 contains two units with a CpG motif. Constructs with greater multiples of the CpG motif may also be made and are considered part of the present invention.

While the Pur-Pur-C-G-Pyr-Pyr motif is the most common motif for the CpG motif, those of ordinary skill in the art will understand that the CpG motif has been known to take other forms as well. One such previously disclosed motif is Pur-Pur-C-G-Pyr-Pur-C-G-Pyr-Pyr. Some non-limiting examples of CpR-ODNs which have been used in the literature and may also be used in the present invention include: TCCATGACGTTCCTGACGTT (Brazolot Millan et al, 1998), TCTCCCAGCGTGCGCCAT (Weiner et al, 1997), GAGAACGCTCGACCTTCGAT (Weiner et al, 1997), TCTCCCAGCGTGCGCCAT (Wooldridge et al, Blood, 89:2994-2998 (1997), TCGTCGTTTTGTCGTTTTGTCGTT (Hartmann et al, PNAS 96:9305-9310 (1999), TCGTCGTTCCCCCCCCCCCC (Hartmann et al (1999).

Preferably, the oligonucleotides are synthesized with a phosophorothioate modified backbone to improve their nuclease resistance.

The art of DNA vaccination is well established and those of ordinary skill in the art are aware of amounts and techniques which are commonly used with respect to such vaccination. DNA vaccines may be administered by intramuscular injection of pure plasmid (i.e., naked) DNA, although the DNA may also be given by intradermal injection or coated onto microscopic gold particles that are introduced biolistically with a gene-gun into cells of the epidermis, all as is well-known in the art. The CpG motifs are preferably unmethylated as its activity as a vaccine may be lost if the CpG motif is methylated. The gene-gun administration approach may be preferred as it has been reported to be associated with a relatively stronger Th2 response to the antigen, whereas i.m. injection of DNA vaccines is associated with a Th1 response (Raz et al 1996). Also as is known, the technique of DNA vaccination may include a postimmunization at an appropriate time following the initial administration, such as, for example, 18 days following the initial injection, or a more substantial period thereafter, such as 12 weeks.

The amounts of DNA to be used in the vaccine are also well-known to those of ordinary skill in the art and can be readily optimized by empirical observation. The amount is preferably between about 1 μg to about 500 μg, although amounts outside of this range may also be used in appropriate circumstances.

### EXAMPLES

The invention is exemplified by the following non-limitative examples:

Methods

Mice

Female mice of the NOD/LtJ strain were raised and maintained under pathogen-free conditions in the Animal Breeding Center of The Weizmann Institute from breeders kindly supplied by Dr. E. Leiter of Jackson Laboratories. These mice manifest insulitis beginning at about one month of age, which progresses to overt hyperglycemia beginning at about three months of age. The cumulative incidence of IDDM rises to 85% or greater by six months of age. Female BALB/c mice were also raised in the Weizmann Institute.

Construction of DNA Vaccine

The DNA vaccine was constructed using the pcDNA3 vector (Invitrogen, NV, Leek, The Netherlands). This is a well-known general purpose cloning and expression vector containing the CMV immediate-early promoter, a polylinker and the bovine growth hormone polyadenylation site. This vector also expresses neomycin resistance in eukaryotic cells. Its restriction map and nucleotide sequence have been published. This sequence is set forth herein as SEQ ID NO:1.

The full length cDNA of human the hsp60 gene was cloned into the pcDNA3 vector under the control of the human cytomegalovirus (CMV) promoter. In brief, hsp60 cDNA in pGEM was amplified by using specific oligonucleotides containing restriction sites for the enzymes BamHI or HindIII. The amplicon and the pcDNA3 vector were purified and digested with BamHI/HindIII. The digested PCR product coding for Hsp60 and the linearized pcDNA3 vector were ligated using T4 DNA ligase, according to the standard protocol given by the manufacturer. The ligated plasmid was transformed into Escherichia coli, and later, sequenced to confirm correct insertion of the cDNA (data not shown).

Plasmid Preparation and Injection

Large-scale plasmid DNA preparations were produced by the alkaline lysis method using Qiagen Plasmid Mega Prep (Qiagen, Santa Clarina, Calif., USA). DNA was ethanol precipitated and resuspended in sterile PBS. Spectrophotometric analysis revealed 260/280 nm ratios≧1.80. Purity of DNA preparations was confirmed on a 1% agarose gel. Endotoxin levels were checked by Limulus Amoebocyte Lysate and always found to be under acceptable levels for in vivo use (less than 0.02 EU/μg DNA).

Eight-week-old NOD or BALB/c females were injected with 100 μl of 10 mM cardiotoxin (Sigma, Rehovot, Israel) into the tibialis anterior muscle using a sterile 27G syringe, fitted with a plastic collar to limit needle penetration to 2 mm. Five, twelve and nineteen days later, the mice were injected with 100 μl, 1 μg/μl, of the desired DNA vaccine, or with PBS as controls.

Phosphorothioate oligonucleotides were synthesized at the Oligonucleotide Synthesis Unit of the Weizmann Institute of Science. One hundred microliters (1 μg/μl) of each preparation were injected as above, following the same time schedule. The oligonucleotide CpG contains two 9 mer segments, which are present in the pcDNA3 ampicilin resistance gene. The control oligonucleotide GpC displays the same nucleotides with an inverted motif.

| | |
|---|---|
| Oligonucleotide CpG: | |
| 5′-TCCATAACGTTGCA-AACGTTCTG-3′. | (SEQ ID NO:2) |
| Oligonucleotide GpC: | |
| 5′-TCCATAAGCTTGCAAAGCTTCTG-3′. | (SEQ ID NO:3) |

Blood Glucose

Hyperglycemia was defined as a blood glucose level exceeding 13 mM, tested using a Beckman Glucose Analyzer II (Beckman Instruments, Brea, Calif., USA).

Peptides and Antigens

Peptides were synthesized by a standard Fmoc procedure, as described (Elias et al, 1994). The peptides were purified by reverse-phase HPLC and their compositions were confirmed by amino acid analysis. The Hsp60 peptide analog denoted p277(Val⁶-Val¹¹), used in this study has the following amino acid sequence: VLGGGVALLRVIPALDSLTPANED (SEQ ID NO:4). This analog of Hsp60 was disclosed in U.S. Pat. No. 6,180,103. Another Hsp60 analog denoted herein p12 has the sequence: EEIAQVATISANGDKEIGNI (SEQ ID NO:5). This analog was disclosed in U.S. Pat. No. 6,110,746. Insulin and Glutamic Acid Decarboxylase (GAD) were purchased from Sigma (Sigma, Rehovot, Israel). Recombinant Hsp60 was prepared as described Elias et al, 1991). Concanavalin A was purchased from Sigma.

T Cell Proliferation

Groups of 8-week-old female NOD mice received three weekly injections of PBS, pcDNA3 or pHsp60, as described. Four weeks after the last dose, the spleens were removed and the T-cell proliferative responses were assayed in vitro in response to the T-cell mitogen Con A, the p277(Val⁶-Val¹¹) peptide or the Hsp60 protein (Elias et al, 1999). Dose-response curves were done to establish optimal doses (not shown). The concentration of 10 μg/ml was chosen for the Hsp60 protein, 1 μg/ml was chosen for p277(Val⁶-Val¹¹), and 1.25 μg/ml for Con A to illustrate the results because these concentrations produced the optimum response. T-cell responses were detected by the incorporation of [methyl-³H]thymidine added to the wells in quadruplicate cultures for the last 18 hours of a 72 hour culture. The stimulation index (SI) was computed as the ratio of the mean c.p.m. of antigen- or mitogen-containing wells to control wells cultured without either. The SD from the mean c.p.m. were always <10%. Background c.p.m. in the absence of antigens, was 800-1500 c.p.m.

Cytokine Assays

Spleen cells were prepared from 10-week-old NOD females. The spleen cells were incubated in triplicate with medium alone, or with increasing concentrations of the CpG or the GpC oligonucleotides. Supernatants were collected at 48 hrs. Cytokines in supernatants were detected by ELISA using Pharmingen paired antibodies (Pharmingen, San Diego, Calif.), according to the Pharmingen cytokine ELISA protocol. Pharmingen recombinant mouse cytokines were used as standards for calibration curves. The concentrations of cytokines are shown as the mean ng/ml derived from calibration curves using recombinant cytokines as standards.

ELISA Assay

Mouse sera were tested for antibodies binding to the p277(Val⁶-Val¹¹) peptide or to Hsp60 as described (Elias et al, 1997). Briefly, 10 μg/ml of the various antigens were applied to assay microplates (Maxisorp:Nunc, Roskilde, Denmark), and the plates were incubated with the test sera. The binding of antibodies was detected using alkaline phosphatase-conjugated anti mouse IgG, or isotype-specific anti-mouse IgG1, IgG2a or IgG2b (Jackson ImmunoResearch). A significant amount of antibody was defined as an OD 405 nm reading higher than 0.25, which is 3 SD above the mean ELISA reading obtained using the sera of ten normal BALB/c mice.

Pancreas Histology

Mice from each treatment group were killed at the age of six months, when almost all the non-treated mice or control-treated NOD mice were sick. The pancreata were fixed in 10% buffered formalin, cut and stained by standard hematoxylin and eosin (H&E), and the average degree of insulitis was assessed over 20 islets scored per pancreas. The islets where classified as clear, when no infiltrate was detected; mildly infiltrated, when peri-insulitis or an intra-islet infiltrate occupying less than 25% of the islet were detected; infiltrated, when 25-50% of the islet was occupied by intra-islet inflammatory cells; and heavily infiltrated, when more that 50% of the islet was occupied.

Statistical Significance

The InStat 2.01 program was used for statistical analysis. Student's t-test and the χ²-test were carried out to assay significant differences between experimental and control groups.

Results

Hsp60 DNA Specifically Immunizes BALB/c Mice.

To test whether the pcDNA3 containing human Hsp60, here named pHsp60, was specifically immunogenic, female BALB/c mice were injected twice (days 5 and 23) i.m., with 100 μg of pcDNA3 or pHsp60, and assayed periodically for serum antibodies.

FIG. 1A shows that the BALB/c mice immunized with pHsp60 developed specific anti-Hsp60 IgG antibodies, whereas no antibodies to the Hsp60 protein could be detected in those animals immunized with pcDNA3. Groups of five 8-week-old female BALB/c were pretreated with cardiotoxin (day 0) and immunized i.m. on days 5 and 23 with pHsp60, pcDNA3, or PBS, or were left untreated. The arrows indicate the time of injections. Serum samples were taken before treatment with cardiotoxin, and ten days after each injection, and antibodies to Hsp60 (FIG. 1A), and to GST (FIG. 1B) were measured by ELISA. The antibodies to GST are shown ten days after the last injection. The means±SD are shown (a single asterisk denotes P<0.02 compared to pcDNA3 treated mice, double asterisk denotes P<0.005 compared to pcDNA3 treated mice, a plus sign denotes P<0.05 compared to pHsp60 treated mice after the first dose of DNA).

Anti-Hsp60 specific antibodies were detected as early as 14 days after a single DNA injection (p<0.02 in comparison to pcDNA3 vaccinated controls). A booster effect was evident ten days after the second DNA injection (p<0.05 in comparison to the same group after the first dose, p<0.005 compared to pcDNA-vaccinated mice). The immune response induced by DNA vaccination with pHsp60 was specific; pHsp60 did not induce antibodies to the non-related recombinant protein Glutathion S-Transferase (GST), as shown in FIG. 1B. These results demonstrate that the pHsp60 construct, but not the empty pcDNA3 vector, can induce in BALB/c mice significant amounts of specific antibodies after one vaccination, and increasing titers after boosting.

DNA Injection Inhibits the Development of NOD Diabetes

To test whether immunization with pHsp60 might modulate the development of spontaneous diabetes in NOD mice, the present inventors vaccinated groups of eight-week old female NOD mice, three times at weekly intervals, and followed their glucose levels. FIG. 2 shows the cumulative incidence of diabetes. Female NOD mice were allocated to groups of 17-18 mice each, and were immunized with PBS, pcDNA3 or pHsp60. A control group was left untreated. The pcDNA3 and Hsp60 vaccinated groups developed a significantly lower incidence of diabetes (a single asterik denotes P<0.001 compared to PBS treated mice, double asterisk denotes P<0.002 compared to PBS treated mice).

It can be seen that both untreated animals and those treated with PBS developed the expected incidence of diabetes for NOD females; around 90% of them were sick by the age of six months. Those vaccinated with pHsp60, and surprisingly, also those vaccinated with the empty pcDNA3 construct showed a significant reduction in the incidence of diabetes. Only about 41% of those treated with pHsp60 (p<0.002) and 38% of those treated with pcDNA3 (p<0.001) were diabetic at the age of six months. Thus DNA vaccination modulates the onset of diabetes by a mechanism that is not associated with the presence of the hsp60 gene in the administered vector.

At the end of the observation period, when the mice were six months old, the pancreata where obtained for histological examination. FIG. 3 shows that 40-50% of the islets obtained from the non-treated or PBS treated mice were heavily infiltrated, and only 5-10% of the islets were free from insulitis. In contrast, 50-70% of the islets obtained from DNA-treated mice were free from insulitis: p<0.001 both for the pcDNA3 injected mice and for the pHsp60 group, compared to non-treated mice or to those treated with PBS. The differences between the groups treated with pHsp60 and pcDNA3 were not significant.

Eight-week-old NOD females were injected with PBS, pcDNA3 or pHsp60 as described in the legend to FIG. 2, or left untreated and their pancreata were removed at the age of six months. The degree of insulitis was determined by scoring at least 20 islets in each pancreas. The islets are depicted as clear (open bars), peri-insulitis or an intra-islet infiltrate occupying less than 25% of the islet (light gray bars), an intra-islet infiltrate occupying 25-50% of the islet (dark gray bars), and an intra-islet infiltrate occupying more than 50% of the islet (black bars). The single asterisk denotes P<0.001 compared to PBS treated mice.

Therefore, DNA vaccination, either with a vector encoding human Hsp60 (pHsp60) or with an empty vector (pcDNA3), diminished the incidence of spontaneous diabetes in NOD females. This effect was accompanied by a significant increase in the number of pancreatic islets remaining free of insulitis.

Inhibition of T cell Proliferation to Hsp60 and to p277(Val⁶-Val¹¹) in DNA-Vaccinated Mice

The process leading to the onset of diabetes in NOD mice can be arrested by administration of peptide p277, derived from the Hsp60 protein (Elias et al, 1991). Successful treatment of NOD mice with peptide p277 or its analog p277(Val⁶-Val¹¹) is associated with the induction of specific antibodies to p277, along with a decrease in the proliferation of T cells to Hsp60 and to p277 (Elias et al, 1997). The present inventors therefore assayed the splenocytes isolated from the DNA-vaccinated, or PBS-treated NOD mice to check their proliferative responses to p277(Val⁶-Val¹¹) and Hsp60. Groups of five 8-week-old female NOD mice received three weekly injections of PBS, pcDNA3 or pHsp60. Four weeks later, their spleens were removed and the T-cell proliferative responses were assayed after 72 hours of stimulation with 10 μg/ml of human Hsp60 (FIG. 4A) or 1 μg/ml of p277(Val⁶-Val¹¹) (FIG. 4B). The results are expressed as the stimulation index (SI)±SD in comparison to paired samples incubated with media alone. (a single asterisk denotes P<0.01 compared to PBS treated mice, a plus sign denotes P<0.05 compared to PBS treated mice).

As shown in FIGS. 4A and 4B, PBS-treated NOD mice manifested spontaneous reactivities to Hsp60 (FIG. 4A) and to p277(Val⁶-Val¹¹) (FIG. 4B). In contrast, splenocytes from the mice vaccinated with pcDNA3 or pHsp60 showed diminished reactivities to p277(Val⁶-Val¹¹) (p<0.05) and to Hsp60 (p<0.01). However, the T cells from both the treated and the non-treated mice showed similar reactivities to Con A (not shown), thus indicating that there was no general inhibition of T cell reactivity induced by DNA vaccination. These results suggested that treatment with plasmid DNA down-regulated the spontaneous proliferative response directed to Hsp60 and p277(Val⁶-Val¹¹) characteristic of the diabetogenic process in NOD mice.

Induction of Antibodies to p277(Val⁶-Val¹¹) and to Hsp60 by DNA Vaccination

The decrease in T-cell proliferation to Hsp60 and its peptide p277 or its analog p277(Val⁶-Val¹¹) observed in NOD mice protected from diabetes by treatment with p277 or its analog is associated with the induction of antibodies directed to p277 (Elias et al, 1997). To see if the protective effect of DNA vaccination might be associated with the appearance of antibodies to Hsp60 and to p277(Val⁶-Val¹¹), the present inventors analyzed antibody responses in DNA-vaccinated animals 14 days after the last DNA injection, at the age of 14 weeks. Groups of 18 NOD mice were treated with PBS, pcDNA3, pHsp60, or CpG or GpC oligonucleotides, while one group was left untreated. Two weeks after treatment, individual sera were tested at a 1:100 dilution for the presence of specific antibodies. FIG. 5A shows serum antibodies to Hsp60 and to p277(Val⁶-Val¹¹), and FIG. 5B shows serum antibodies to GAD, insulin and GST. Data represent the mean±SD for each group (the asterisk denotes P<0.001 compared to PBS treated mice).

FIG. 5A shows that antibodies to p277(Val⁶-Val¹¹) Were not detected in the sera of untreated or PBS-injected animals. The absence of antibodies to p277(Val⁶-Val¹¹) and to Hsp60 is expected in NOD mice of this age (Krause et al, 1999). Antibodies to p277(Val⁶-Val¹¹) in BALB/c mice immunized with pHsp60 were not detected, where the appearance of anti-Hsp60 antibodies was demonstrated (FIG. 7 and data not shown). However, NOD mice vaccinated with pHsp60 or with pcDNA3 manifested significant levels of antibodies to p277(Val⁶-Val¹¹) (p<0.001). Thus, inhibition of diabetes in NOD mice by DNA vaccination with either pcDNA3 or pHsp60 is associated with the induction of antibodies to Hsp60 and to the peptide p277(Val⁶-Val¹¹), even though the pcDNA3 construct does not contain genetic material encoding Hsp60.

CpG Injection Induces Antibodies to Hsp60 and to Peptide p277(Val⁶-Val¹¹)

Bacterial DNA contains immunostimulatory sequences that are recognized by the immune system as danger signals, and trigger a series of responses in cells of both the innate and adaptive immune system (Lipford et al, 1998; Krieg et al, 19989; Krieg, 1999). The pcDNA3 vector contains the immunostimulatory CpG sequence in its ampicilin resistance gene (Boccaccio et al, 1999). The present inventors tested whether a DNA oligonucleotide with two CpG sequences could induce the production of specific antibodies to Hsp60 and to p277(Val⁶-Val¹¹) that followed vaccination with pcDNA3. As a control the oligonucleotide GpC was used, in which the CpG motifs were inverted.

Eight-week old NOD mice were treated with the oligonucleotides CpG or GpC, and antibodies to Hsp60, p277(Val⁶-Val¹¹), GAD, insulin and GST were assayed by ELISA at the age of 14 weeks. As shown in FIG. 5A, treatment with the CpG oligonucleotide induced significant levels of antibodies to Hsp60 and to p277(Val⁶-Val¹¹) (p<0.002). Moreover, the titer of antibodies induced by CpG was also significant when compared to the levels found in GpC treated mice (p<0.02). Since the GpC oligonucleotide failed to induce specific antibodies to Hsp60 or to p277(Val⁶-Val¹¹), the induction of these specific antibodies by the pcDNA3 vector may be linked to the presence of the CpG motif. Thus, stimulation of the NOD immune system with an immunostimulatory sequence alone can trigger the production of specific autoantibodies to Hsp60 and its peptide analog p277(Val⁶-Val¹¹).

It was conceivable that the appearance of antibodies to p277(Val⁶-Val¹¹) and to the Hsp60 antigen reflected a polyclonal activation of IgG-secreting clones. Therefore, the sera were assayed from the different groups of mice for antibodies to insulin, GAD and the bacterial recombinant protein GST. FIG. 5B shows that the levels of antibodies to insulin, GAD or GST were essentially the same among the groups. Thus, administration of the pHsp60, the pcDNA3 vector, or of the CpG oligonucleotide induced specific antibodies to Hsp60 and to p277(Val⁶-Val¹¹). This indicates that the induction of specific antibodies to Hsp60 and to p277(Val⁶-Val¹¹) was not the result of polyclonal activation.

CpG Injection Inhibits NOD Diabetes

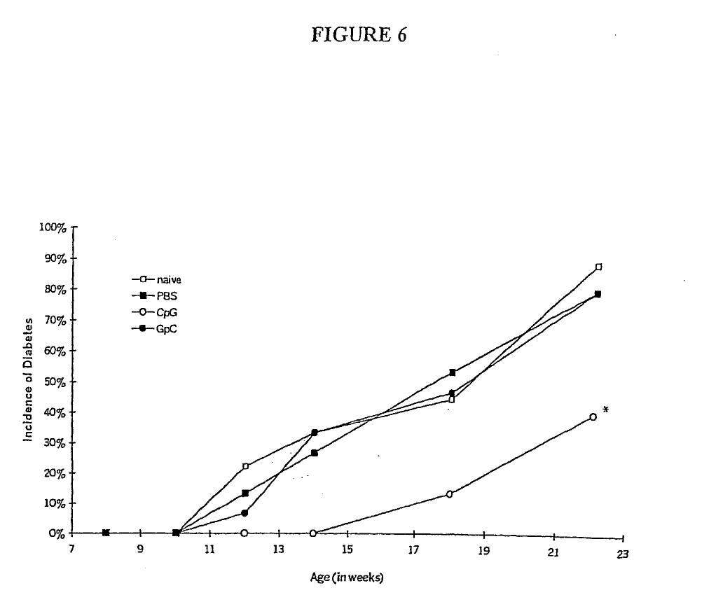

To test whether administration of the CpG oligonucleotide can, like the pcDNA3 vector, modulate the development of spontaneous diabetes in NOD mice, groups of eight-week old female NOD mice were vaccinated three times at weekly intervals, and followed their glucose levels. FIG. 6 shows the cumulative incidence of diabetes. Female NOD mice were allocated to groups of 15-18 mice each, and were immunized with PBS, CpG or GpC. A control group was left untreated. The CpG vaccinated group developed a significantly lower incidence of diabetes (the asterisk denotes P<0.015 compared to GpC treated mice).

It can be seen that both untreated animals and those treated with PBS developed the expected incidence of diabetes for NOD females; around 85% of them were sick by the age of six months. Furthermore, the incidence of diabetes was not affected in the group of mice vaccinated with the control oligonucleotide GpC. However, the mice vaccinated with CpG showed a significant reduction in the incidence of diabetes. Only about 40% of those treated with CpG (p<0.015) were diabetic at the age of six months.

Therefore, the protective effect observed after immunization with pcDNA3 could be reproduced with a DNA oligonucleotide containing CpG motifs. The mechanism involved is sequence specific, since the control oligonucleotide GpC did not have a significant effect on the incidence of the disease.

Antibody Isotypes

The isotype of specific serum antibodies characterizes the phenotype of the immune response to an antigen; the antibody isotype reflects the in vivo integration of the complex network of cytokines that regulates the immune response. Antibodies of the IgG1 and IgG2b isotypes evidence a specific Th2 response, because they are dependent on IL-4 and TGF-α, respectively (McIntyre et al, 1993; Snapper et al, 1993). In contrast, antibodies of the IgG2a isotype are IFN-γ dependent, and they reveal the existence of a Th1 response (McIntyre et al, 1993; Snapper et al, 1993). Therefore, the isotypes of the antibodies to p277(Val⁶-Val¹¹) and to Hsp60, detected in DNA-vaccinated mice 14 days after the last injection, were studied. The isotypes of serum antibodies to Hsp60 (FIG. 7A), or p277(Val⁶-Val¹¹) (FIG. 7B) from NOD (black bars, n=18) or BALB/c (white bars, n=5) mice treated with pcDNA3, pHsp60 or the CpG oligonucleotide were determined two weeks after the last vaccination. The isotypes of the antibodies were tested at a 1:100 dilution of individual sera. Data are shown as the mean±SD for each group (the asterisk denotes P<0.01 compared to IgG2a levels in the same group).

FIGS. 7A and 7B show that the antibodies induced to Hsp60 and to p277(Val⁶-Val¹¹) were predominantly of the IgG2b isotype (p<0.01 in comparison to IgG2a levels). There was also a slight increase in the levels of IgG1 antibodies to Hsp60 and p277(Val⁶-Val¹¹), but this induction was significant in comparison to the amount of the IgG2a specific antibodies only in the group treated with the CpG oligonucleotide. Furthermore, there were no differences in the isotypes of the antibodies between the pHsp60, pcDNA3 and CpG treated NOD mice. Thus, the inhibition of diabetes induced by the DNA plasmids or by the CpG oligonucleotide in both cases was accompanied by the induction of antibodies to Hsp60 and p277(Val⁶-Val¹¹) of the IgG2b isotype, characteristic of a Th2-type response.

Interestingly, there was a marked difference in the antibodies induced in the BALB/c compared to the NOD mice. The BALB/c mice made antibodies to Hsp60 when they were vaccinated with pHsp60 but not following immunization with pcDNA3 (FIG. 1A). Moreover, the antibodies induced were mainly of the IgG1 subclass, and the BALB/c mice did not make antibodies to p277(Val⁶-Val¹¹). These results indicate strain-specific differences in the cytokine networks that regulate antibody secretion to the self antigen Hsp60.

Induction of IL-10 and IFN_{γ} Secretion by the CpG Oligonucleotide

To gain some insight into the cytokine effects of CpG, the amounts of IL-10, a Th2 cytokine, and IFN_{γ}, a Th1 cytokine, secreted by NOD spleen cells after CpG oligonucleotide stimulation in vitro, were assayed. Since different cytokines are secreted in different physiological amounts, included control groups of spleen cells incubated with ConA, a prototypic T-cell mitogen, were included. NOD spleen cells were incubated in triplicates with increasing concentrations of the CpG or GpC oligonucleotides for 48 hours, and their supernatants were tested for the amounts of IFN_{γ}, of IL-10 cytokine released. Control spleen cells were incubated with Con A, 1,25 μg/ml, to obtain a relative response magnitude. FIG. 8A shows IL-10 production, and FIG. 8B shows IFN_{γ} production. The data are shown as the mean ±SD of triplicates. Three independent experiments produced similar results.

As shown in FIGS. 8A and 8B, the CpG oligonucleotide induced both IL-10 and IFN_{γ} production in NOD spleen cells in a dose-dependent manner. However, when compared to the amount of cytokine released in response to Con A stimulation, the effect of CpG treatment seemed to be relatively more effective in stimulating IL-10 than in stimulating IFN_{γ}. CpG-triggered a maximal release of IFN_{γ} of 7 ng/ml, about one-fourth of the IFN_{γ} released by Con A. In contrast, CpG induced the release of 1.5 ng/ml of IL-10, almost 10 times higher than the amount induced by Con A stimulation.

CpG Activates Spleen Cells In Vitro

In order to study the regulatory mechanisms involved in control of spontaneous NOD diabetes by CpG treatment, we used oligonucleotides containing one or two CpG motifs. The oligos were respectively called DP (double positive) or SP (single positive). As controls we used oligos where the CpG motifs were inverted and therefore rendered inactive. These control oligos were called DN (double negative) and SN (single negative). To check the in vitro effects of CpG motifs on spleen cells, splenocytes were prepared from normoglycemic 3-month old NOD females and incubated for 72 hrs in 96-well plates with different concentrations of control, or CpG-containing oligonucleotides. During the last 16 hrs., labelled thymidine was added to the culture medium, and at the end of the incubation period, the cells were harvested and the proliferation in response to the different stimuli was quantified. It was found that oligonucleotides containing CpG motifs (DP and SP) induce a dose-dependent proliferation. Moreover, the oligo that contains two CpG motifs (DP) induces stronger proliferations than the oligo containing a single CpG motif (SN). Control oligonucleotides were the CpG motif has been removed by inversion (DN and SN) had no significant effect. CpG induced responses are as strong as those induced with LPS. Irradiation of the splenocytes with 3000 Rads (the standard procedure for their use as APCs in the stimulation of T-cell lines in culture) abrogated CpG-induced proliferation.

The same experiment was performed with spleens taken from NOD females at different ages. No differences were detected in the proliferative responses induced in response to CpG stimulation, either SP or DP. Furthermore, no significant differences were seen when NOD spleens were compared to C57BL/6 or BALB/c spleens taken from age and sex-matched animals. Therefore, it was confirmed that CpG induces proliferation of spleen cells, and this proliferation is inhibited by gamma irradiation.

CpG Upregulates Hsp60 Expression

Western blot experiments were performed to check if CpG stimulation upregulates the expression of Hsp60 on splenocytes. NOD spleen cells were isolated from normoglycemic females and stimulated in vitro for 48 hrs. with different concentrations of the DP oligo. Stimulated splenocytes were then lysed on ice for 5 minutes, and after 10 minutes of centrifugation at 14000 rpm, the supernatants (representing the cytoplasmic fraction) were analysed by Western blot using anti Hsp60 specific polyclonal antibodies. Incubation with CpG induced the expression of Hsp60 in a dose dependent manner. Therefore, the effect of CpG in inhibiting diabetes could involve the up-regulation of Hsp60 by CpG.

CpG Induces Secretion of Hsp60

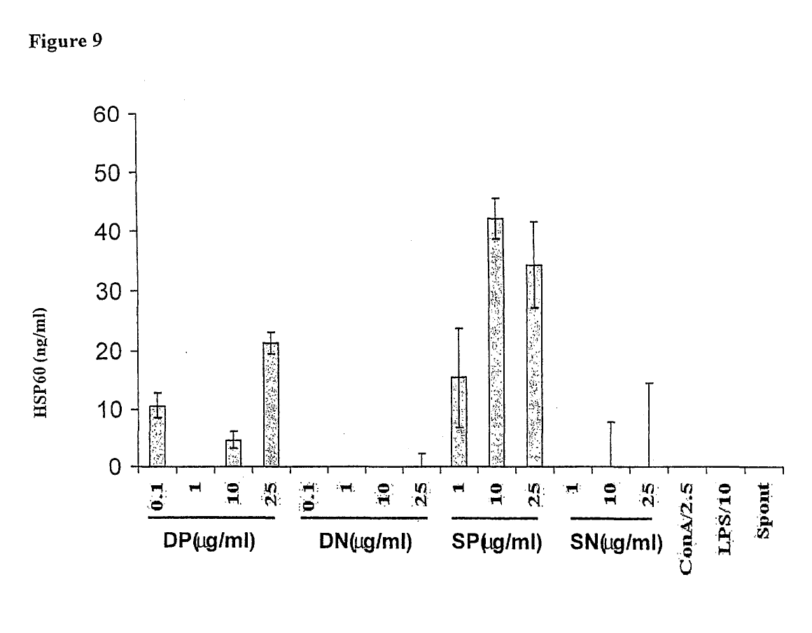

The release of Hsp60 to tissue culture medium after stimulation of splenocytes by CpG was studied. NOD splenocytes were stimulated in vitro with CpG-positive (DP and SP) or control (DN and SN) oligos for 48 hrs. A capture Elisa method was used to quantify Hsp60 present in tissue culture supernatants at the end of the stimulation period. Hsp60 can, indeed, be detected in a dose-dependent manner in supernatants of splenocytes activated with CPG as presented in FIG. 9). Spleen cells activated with Con A or LPS do not release Hsp60, suggesting that release of Hsp60 is a specific feature of the CpG/TLR-9 pathway, not shared with other pathways leading to T or B cell activation, even when they also signal through pattern recognition receptors (LPS/TLR-4).

CpG Activates Hsp60-specific T-Cell Lines

CpG-containing oligonucleotides upregulated Hsp60 expression, and release into extracellular medium; the effect of CpG on Hsp60-specific T-cell lines in the presence of irradiated APCs was tested. NOD T-cell lines raised against two immunodominant epitopes of mammalian Hsp60, p12 and p277, and an NOD T-cell line specific to OVA as a control were used. Table 1 show that CpG-containing oligos induced T-cell proliferation. Furthermore, the number of CpG sequences present in the oligo also influenced the strength of the proliferation, since DP oligos induced stronger proliferations than did the SP oligos. No significant proliferation was observed in response to the control oligos DN and SN. The OVA-specific T-cell line did not proliferate in response to the CpG positive oligos, but all the lines proliferated in response to their appropriate antigen. LPS did not induce a significant proliferation in the presence of APCs, indicating that the T-cell line is not contaminated with non-T cells. These CpG-induced responses were inhibited by anti-MHC-class II antibodies, indicating that CpG induced proliferation involves presentation of Hsp60 epitopes in the MHC-class II molecule. Therefore, it can be concluded that CpG up-regulates Hsp60 and induces the presentation of the Hsp60 epitopes p277 and p12, that can be presented to specific T-cells and can stimulate them to proliferate.

TABLE 1
CpG stimulates mammalian Hsp60-specific T-cell lines.

| | T-cell proliferation (SI) | | | | |
|---|---|---|---|---|---|
| Clones | DN | DP | SN | SP | Antigen |
| Anti-p12 | 3 ± 0.5 | 19 ± 2.6 | 2.5 ± 0.4 | 9.5 ± 1.2 | 41 ± 1.9 |
| Anti-p277 | 1.5 ± 0.1 | 18 ± 1.9 | 1.4 ± 0.2 | 12 ± 1.5 | 83 ± 6.1 |
| (Val⁶-Val¹¹) | | | | | |
| Anti-OVA | 2.2 ± 0.3 | 2.1 ± 0.2 | 2.3 ± 0.5 | 3.1 ± 0.4 | 30 ± 3.6 |

NOD T cell lines specific to p277(Val⁶-Val¹¹), p12 or OVA were stimulated in vitro with CpG positive or control oligos (10 μg/ml), or with their corresponding antigens (10 μg/ml). Proliferation was measured after 72 hrs. and is expressed as stimulation index (SI).

CpG Shifts the Cytokines of Activated T-Cells to an Anti-Inflammatory Profile

The production of cytokines in response to stimulation with CpG-containing oligonucleotides was studied. As shown in Table 2, in vitro stimulation with oligos containing one or two CpG motifs induced the release of IL-10 and IFN_{γ}. IL-2 or IL-4 in the supernatants of CpG-stimulated T-cell lines was not detected. When compared to the amount of cytokine released in response to peptide-specific stimulation, CpG induced secretion of higher amounts of IL-10 and lower amounts of IFN_{γ}. The relative increase in IL-10 secretion might explain the protective effect of CpG on NOD diabetes, because IL-10 is known to have suppressor effects on the immune response (Akidis and Blaser, 2001).

TABLE 2
CpG-mediated T-cell activation leads to IL-10 and IFNγ secretion.

| | Cytokine release (pg/ml) | | | | | | | |
|---|---|---|---|---|---|---|---|---|
| | Medium | Con A | LPS | Antigen | DP | DN | SP | SN |
| IL-10 | 0 ± 6 | 1200 ± 45 | ND | 720 ± 32 | 2300 ± 134 | 365 ± 27 | 2800 ± 235 | 340 ± 10 |
| IFNγ | ND | 70 ± 8 | 11 ± 3 | 114 ± 12 | 43 ± 6 | 19 ± 3 | 37 ± 4 | 21 ± 4 |

P12-specific T-cells were activated in the presence of APCs for 72 hrs. with LPS (10 μg/ml), Con A (1.25 μg/ml), CpG positive and control oligonucleotides (10 μg/ml) or p12 (10 μg/ml). The supernatants were collected and secreted cytokines were measured by capture ELISA. The results are expressed as pg/ml of cytokine secreted. ND, not detected.

CpG Down-Regulates APC Function In Vivo

Experiments were performed to test whether CpG-treatment influences APC function in vivo. It was recently reported that LPS down-regulates APC function in NOD mice (Tian et al., 2001). Three-month old females were injected once with 100 μg/mouse of an oligonucleotide containing two CpG, or a control oligonucleotide containing two inactive GpC motifs. Splenocytes were prepared from oligonucleotide- or PBS-treated animals, and then irradiated with 3000 Rads in order to use them as APCs. The APCs were coincubated with the NOD T-cell line specific for the p12 peptide of Hsp60, and then the line (in the presence of the different APCs) was stimulated either with the p12 peptide, or with the CpG or control oligonucleotides. T-cells activated in the presence of 10 μg/ml of p12 and APCs prepared from CpG treated mice showed significantly lower SI (150±13) than those activated in the presence of APCs isolated from PBS or GpC treated mice (298±24 and 275±18, respectively). Therefore, APCs prepared from CpG-treated mice are less efficient than those taken from GpC or PBS treated mice. There were no significance differences in the proliferative responses induced by CpG containing oligonucleotides.

In order to better understand the mechanisms underlying this apparent down-regulation of APC function, we studied the cytokines released in response to stimulation with p12 or CpG-containing oligonucleotides in the presence of the different APC preparations.

TABLE 3
APCs isolated form CpG-treated mice induce the release of
higher levels of IL-10 and IL-5 in response to peptide stimulation.

| | Cytokine secreted (pg/ml) | | |
|---|---|---|---|
| Cytokine | PBS-APC + p12 | GpC-APC + p12 | CpG-APC + p12 |
| IL-4 | ND | 18 ± 4 | 65 ± 13 |
| IL-5 | 4300 ± 326 | 4253 ± 183 | 4156 ± 68 |
| IL-10 | ND | 35 ± 5 | 83 ± 12 |
| IFNγ | 7225 ± 658 | 7465 ± 425 | 7736 ± 397 |

APCs were isolated from animals treated with oligos containing or not CpG motifs and used to stimulate a p12 specific T-cell line. After 72 hrs. of stimulation, tissue culture supernatants were collected and released cytokines were quantified using a capture Elisa.

Table 3 shows that there were no differences in the levels of IFN_{γ} or IL-5, although there was a dose-dependent release of IL-10, a well known suppressor cytokine, and IL-4 when the p12 line was incubated with APCs taken from CpG treated mice. Therefore, CpG treatment affected APC function, leading to the generation of APCs with diminished stimulatory properties. This effect seems to be mainly mediated mainly by the secretion of IL-10, a suppressor cytokine, and also by IL-5 and IL-4.

CpG Down-Regulates Spontaneous NOD Autoimmunity

In view of the effects of CpG in vitro, experiments were designed to reveal the in vivo effects of CpG on the specific autoimmunity of NOD mice related to diabetes. An oligo containing two CpG motifs was injected once (100 μg/mouse) to 2 month old NOD females. Splenocytes were prepared 1 month after treatment with the CpG or the control oligo, and the spontaneous proliferative responses to self-antigens associated with diabetes were studied (GAD, Hsp60 and insulin), and to CpG containing oligos. Table 4 shows that mice treated with the CpG-containing oligo showed decreased proliferations to Hsp60, GAD and Insulin, although no difference was seen in the proliferative response to the oligos themselves (Table 4), or to Con A, p¹², p277(Val⁶-Val¹¹), p34 and p35 (data not shown). Thus, CpG treatment downregulated the spontaneous self-reactivity directed to specific diabetes-associated antigens in NOD mice.

TABLE 4
CpG treatment downregulates NOD spontaneous self-reactivity

| | T-cell proliferation (CPM) | | |
|---|---|---|---|
| In vitro stimulus | PBS | GpC | CpG |
| Hsp60 (25 μg/ml) | 9053 ± 567 | 8967 ± 697 | 6001 ± 364 |
| Insulin (25 μg/ml) | 6397 ± 367 | 6354 ± 654 | 2860 ± 564 |
| GAD (5 μg/ml) | 25684 ± 249 | 23687 ± 3200 | 16588 ± 2799 |
| DP (10 μg/ml) | 35687 ± 4895 | 36479 ± 2156 | 34468 ± 3468 |
| DN (10 μg/ml) | 5640 ± 235 | 6800 ± 563 | 5870 ± 157 |
| SP (10 μg/ml) | 27868 ± 1254 | 28473 ± 2458 | 26498 ± 3284 |
| SN (10 μg/ml) | 10546 ± 2346 | 8703 ± 564 | 9218 ± 1166 |

NOD splenocytes were isolated 1 month after treatment with CpG, GpC containing oligos or PBS and their proliferative responses to self antigens and oligonucleotides were followed in vitro. The results are presented as mean cpm±SD.

CpG Shifts the Cytokine Profile of Spontaneous Autoimmunity

The phenotype of the immune responses to diabetes-associated self-antigens and CpG was studied. Splenocytes were isolated from oligonucleotide or PBS-treated mice, and cytokine release in response to in vitro stimulation was measured by capture-ELISA. Table 5 shows that CpG-treated mice shifted their autoimmune response towards a Th2 phenotype, with increased secretion of IL-10 and a decrease of IFN_{γ} secreted in response to restimulation with diabetes-associated antigens. A significant release of cytokines in response to incubation with the control peptide p35 was not detected. Thus, treatment with CpG shifts the spontaneous autoimmune response of NOD mice from Th1 to Th2.

TABLE 5
CpG treatment shifts spontaneous Th1
self-reactivity to a Th2 phenotype

| | Cytokine secreted | | | |
|---|---|---|---|---|
| In vitro | IL-10 (pg/ml) | | IFNγ(pg/ml) | |
| stimulus | CpG | GpC | CpG | GpC |
| Hsp60 | 721 ± 56 | 115 ± 19 | 78 ± 6 | 658 ± 46 |
| (25 μg/ml) | | | | |
| Hsp60/p277 | 256 ± 35 | ND | ND | 1745 ± 236 |
| (Val⁶-Val¹¹) | | | | |
| (25 μg/ml) | | | | |
| Hsp60/p12 | 864 ± 124 | ND | ND | 420 ± 45 |
| (25 μg/ml) | | | | |
| GAD | 2498 ± 364 | 1496 ± 112 | 1678 ± 214 | 3968 ± 387 |
| (25 μg/ml) | | | | |
| GAD/p34 | 654 ± 94 | ND | ND | 384 ± 26 |
| (25 μg/ml) | | | | |
| GAD/p35 | ND | ND | ND | ND |
| (25 μg/ml) | | | | |
| Insulin | 378 ± 36 | ND | ND | 1647 ± 54 |
| (25 μg/ml) | | | | |

NOD splenocytes were isolated 1 month after treatment with CpG, GpC containing oligos or PBS to follow cytokine release in response to in vitro stimulation with different self-antigens. The results are presented as mean pg/ml of secreted cytokine.

CpG Vaccination for IDDM Patients

CpG vaccination for IDDM patients according to the present invention is tested in several studies. The target population for these studies is newly-diagnosed and established IDDM Patients. In these patients, CpG vaccine is expected to modulate the destructive pro-inflammatory autoimmune attack on the remaining reserve of beta-cells, allowing their survival and continued function. The maintenance of beta-cell function should result in improved metabolic control, reduced insulin requirement and reduced rate of hypoglycemic attacks. Improved metabolic control has been shown to reduce and postpone major Diabetes-related health complications during the later stage of the disease.

Phase I—Safety study in Established IDDM; Double-blind, Randomized, Placebo-controlled: 3 doses of CpG vaccine, 2 administrations; Dosing schedule: 0, 6 months+7 weeks follow-up.

Phase II—Safety & Efficacy study—Established IDDM; Double-blind, Randomized, Placebo-controlled: 3 doses of CpG vaccine, 4 administrations; Dosing schedule: 0, 1, 6 & 12 months+6 months follow-up. Patients with Basal C-peptide level equal or greater than 0.1 pmol/ml are included in this study. Tested endpoints: Stimulated C-peptide, insulin dose, HbA1C, immunological response to CpG vaccine (Th2 shift).

Phase II—Efficacy & Safety—Newly diagnosed IDDM adults; Double-blind, Randomized, Placebo-controlled: 1-3 doses of CpG vaccine, 4 administrations; Dosing schedule: 0, 1, 6 & 12 months+6 months follow-up. Patients with basal C-peptide level equal or greater than 0.1 pmol/ml are included in this study. Tested endpoints: Stimulated C-peptide, insulin dose, HbA1C, immunological response to CpG vaccine (Th2 shift).

Phase II—Efficacy & Safety—Newly diagnosed IDDM children; Double-blind, Randomized, Placebo-controlled: 1-3 doses of CpG vaccine, 4 administrations. Dosing schedule: 0, 1, 6 & 12 months+6 months follow-up. Patients with basal C-peptide level equal or greater than 0.1 pmol/ml are included in this study. Tested endpoints: Stimulated C-peptide, insulin dose, HbA1C, immunological response to CpG vaccine (Th2 shift).

All these clinical studies are based on the working hypothesis that CpG vaccine acts as a vaccine, requiring a limited number of administrations which were timed according to the conventional schedule for vaccines. CpG vaccine may be also administered as a therapeutic vaccine for chronic treatment and that in order to maintain the disease-specific Th1 to Th2 shift a more intensive dosing schedule is required.

The DNA vaccines for treatment of IDDM patients may further comprises DNA sequences encoding a polypeptide selected from the group consisting of Hsp60, p277, p277(Val⁶-Val¹¹) and p12. Additional vaccines and treatment regimens may further comprises administration of a peptide or polypeptide molecule selected from the group consisting of Hsp60, p277, p277(Val⁶-Val¹¹) and p12. The peptide or polypeptide molecule may be administered together with the DNA vaccine or independent or separate from the DNA vaccine.

The foregoing description of the specific embodiments so fully reveals the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Ablamunits et al, “Islet T cells secreting IFN-gamma in NOD mouse diabetes: arrest by p277 peptide treatment”, J Autoimmun 11:73 (1998)

Akdis and Blaser, “Mechanisms of interleukin-10-mediated immune suppression”, Immunology 102: 131 (2001)

Atkinson et al, “The NOD mouse model of type 1 diabetes: as good as it gets?”, Nat med 5:601 (1999)

Boccaccio et al, “Non-coding plasmid DNA induces IFN-gamma in vivo and suppresses autoimmune encephalomyelitis”, Int Immunol 11:289 (1999)

Bockova et al, “Treatment of NOD diabetes with a novel peptide of the hsp60 molecule induces Th2-type antibodies”, J Autoimmun 10:323 (1997)

Birk et al, “NOD mouse diabetes: the ubiquitous mouse hsp60 is a beta-cell target antigen of autoimmune T cells”, J Autoimmun 9:159 (1996)

Brazolot Millan et al, “CpG DNA can induce strong Th1 humoral and cell-mediated immune responses against hepatitis B surface antigen in young mice”, PNAS 95:15553-15558 (1998)

Campbell et al, “Reduction in insulitis following administration of IFN-gamma and TNF-alpha in the NOD mouse”, J Autoimmun 4:249 (1991)

Carpentier et al, “Successful treatment of intracranial gliomas in rat by oligodeoxynucleotides containing CpG motifs ”, Clin Cancer Res 2000, 6(6):1469-73 (2000)

Cohen I., Tending Adam's Garden: Evolving the Cognitive Immune Self, Academic Press, London (2000)

Elias et al, “Vaccination against autoimmune mouse diabetes with a T-cell epitope of the human 65-kDa heat shock protein”, Proc Natl Acad Sci USA 88:3088 (1991)

Elias et al, “Peptide therapy for diabetes in NOD mice”, Lancet 343:704 (1994)

Elias et al, Treatment of autoimmune diabetes and insulitis in NOD mice with heat shock protein 60 peptide p277”, Diabetes 44:1132 (1995)

Elias et al, “Hsp60 peptide therapy of NOD mouse diabetes induces a Th2 cytokine burst and downregulates autoimmunity to various beta-cell antigens”, Diabetes 46:758 (1997)

Elias et al, “Regulation of NOD mouse autoimmune diabetes by T cells that recognize a TCR CDR3 peptide”, Int Immunol 11:957 (1999)

Gilkeson et al, “Modulation of renal disease in autoimmune NAB/NZW mice by immunization with bacterial DNA”, J Exp Med 183:1389 (1996)

Hassett et al, “Immune responses following neonatal DNA vaccination are long-lived, abundant, and qualitatively similar to those induced by conventional immunization”, J Virol 74:2620 (2000)

Hartmann et al, “CpG DNA: A potent signal for growth, activation, and maturation of human dendritic cells”, PNAS 96:9305-9310 (1999)

King et al, “TGF-betal alters APC preference, polarizing islet antigen responses toward a TH2 phenotype”, Immunity 8:601 (1998)

Klinman et al, “CpG motifs present in bacteria DNA rapidly induce lymphocytes to secrete interleukin 6, interleukin 12, and interferon gamma”, Proc Natl Acad Sci USA 93:2879 (1996)

Klinman et al, “Contribution of CpG motifs to the immunogenicity of DNA vaccines”, J Immunol 158:3635 (1997)

Krause et al, “Inhibition of diabetes in NOD mice by idiotypic induction of SLE”, J Autoimmun 13:49 (1999)

Krieg et al, “The role of CpG dinucleotides in DNA vaccines”, Trends Microbiol 6:23 (1998)

Krieg et al, “CpG DNA: a novel immunomodulator [letter]”, Trends Microbiol 7:64 (1999)

Lipford et al, “CpG-containing synthetic oligonucleotides promote B and cytotoxic T cell responses to protein antigen: a new class of vaccine adjuvants”, European Journal of Immunoloy 27:(9)2340-2344 (1997)

Lipford et al, “Bacterial DNA as immune cell activator”, Trends Microbiol 6:496 (1998)

McIntyre et al, “Transforming growth factor beta 1 selectivity stimulates immunoglobulin G2b secretion by lipopolysaccharide-activated murine B cells”, J Exp Med 177:1031 (1993)

Nicoletti et al, “Paradoxical antidiabetogenic effect of gamma-interferon in DP-BB rats”, Diabetes 47:32 (1998)

Raz et al (1996), “Preferential induction of a Th1 immune response and inhibition of specific IgE antibody formation by plasmid DNA immunization”, PNAS 93:5141-5145.

Rothe et al, “IL-18 inhibits diabetes development in nonobese diabetic mice by counterregulation of Th1-dependent destructive insulitis”, J Immunol 163:1230 (1999)

Serreze et al, “Immunostimulation circumvents diabetes in NOD/Lt mice”, J Autoimmun 2:759 (1989)

Sobel et al, “Low dose poly I:C prevents diabetes in the diabetes prone BB rat”, J Autoimmun 11:343

Snapper et al, “Towards a comprehensive view of immunoglobulin class switching”, Immunol Today 14:15 (1993)

Stevenson et al, “DNA vaccination against multiple myeloma”, Semin Hematol 36:38 (1999)

Tascon et al, “Vaccination against tuberculosis by DNA injection”, Nat Med 2:888 (1996)

Tian et al. “Lipopolysaccharide-activated b cells down-regulate th1 immunity and prevent autoimmune diabetes in nonobese diabetic mice” J. Immunology 167:1081 (2001)

Tisch et al, “Insulin-dependent mellitus”, Cell 85:291 (1996)

Waisman et al, “Suppressive vaccination with DNA encoding a variable region gene of the T-cell receptor prevents autoimmune encephalomyelitis and activates Th2 immunity”, Nat Med 2:899 (1996)

Weiner et al, “Immunostimulatory oligodeoxynucleotides containing the CpG motif are effective as immune adjuvants in tumor antigen immunization”, PNAS 94:10833-10837(1997)

Wolff et al, “Long-term persistence of plasmid DNA and foreign gene expression in mouse muscle”, Hum Mol Genet 1:363 (1992)

Wooldridge et al, “Immunostimulatory Oligodeoxynucleotides Containing CpG Motifs Enhance the Efficacy of Monoclonal Antibody Therapy of Lymphoma”, Blood, 89:2994-2998 (1997)

Yang et al, “Effect of tumor necrosis factor alpha on insulin-dependent diabetes mellitus in NOD mice. I. The early development of autoimmunity and the diabetogenic process”, J Exp Med 180:995 (1994)

What is claimed is:

1. A method of treatment of an ongoing autoimmune disease, comprising vaccinating a patient having an autoimmune disease with an effective amount of a DNA vaccine comprising a DNA molecule with a sequence comprising a CpG motif.

2. The method in accordance with claim 1, wherein said autoimmune disease is insulin-dependent diabetes mellitus.

3. The method in accordance with claim 1, wherein said DNA molecule has a sequence comprising the dinucleotide CG flanked at the 5′ side by two purines and at the 3′ side by two pyrimidines.

4. The method in accordance with claim 3, wherein said sequence comprises AACGTT.

5. The method in accordance with claim 2, wherein said DNA vaccine further comprises DNA sequences encoding a peptide or a polypeptide selected from the group consisting of Hsp60, p277, p277(Val⁶-Val¹¹) and p12.

6. The method in accordance with claim 2, further comprising the step of administering a peptide or a polypeptide molecule selected from the group consisting of Hsp60, p277, p277(Val⁶-Val¹¹) and p12.

7. The method of claim 6 wherein the peptide or polypeptide is co-administered with the DNA vaccine.

8. The method of claim 6 wherein the peptide or polypeptide is administered separately from the DNA vaccine.

9. A method for prevention of an autoimmune disease, comprising vaccinating an individual in need of such prevention with an effective amount of a DNA vaccine comprising a DNA molecule with a sequence comprising a CpG motif.

10. The method in accordance with claim 9, wherein said autoimmune disease is insulin-dependent diabetes mellitus.

11. The method in accordance with claim 9, wherein said DNA vaccine has a sequence comprising the dinucleotide CG flanked at the 5′ side by two purines and at the 3′ side by two pyrimidines.

12. The method in accordance with claim 11, wherein said sequence comprises AACGTT.

13. The method in accordance with claim 9, wherein said DNA vaccine further comprises DNA sequences encoding a peptide or a polypeptide selected from the group consisting of Hsp60, p277, p277(Val⁶-Val¹¹) and p12.

14. The method in accordance with claim 9, further comprising the step of administering a peptide or a polypeptide molecule selected from the group consisting of Hsp60, p277, p277(Val⁶-Val¹¹) and p12.

15. The method of claim 14 wherein the peptide or polypeptide is co-administered with the DNA vaccine.

16. The method of claim 14 wherein the peptide or polypeptide is administered separately from the DNA vaccine.

17. In a method of preparing a vaccine for treatment or prevention of an ongoing autoimmune disease the improvement which comprises incorporating in the vaccine a DNA molecule comprising a CpG motif.

18. The method of claim 17 wherein the autoimmune disease is insulin-dependent diabetes mellitus.

19. The method of claim 17, wherein said DNA molecule has a sequence comprising the dinucleotide CG flanked at the 5′ side by two purines and at the 3′ side by two pyrimidines.

20. The method of claim 19, wherein said sequence comprises AACGTT.

21. The method of claim 17, wherein said DNA vaccine further comprises DNA sequences encoding a peptide or a polypeptide selected from the group consisting of Hsp60, p277, p277(Val⁶-Val¹¹) and p12.

22. The method of claim 17, wherein said vaccine further comprises a peptide or a polypeptide molecule selected from the group consisting of Hsp60, p277, p277(Val⁶-Val¹¹) and p12.

23. A DNA vaccine comprising DNA sequences encoding a peptide or polypeptide antigen associated with IDDM.

24. The DNA vaccine according to claim 24, wherein said CpG motif further comprises DNA sequences encoding a peptide or a polypeptide selected from the group consisting of Hsp60, p277, p277(Val⁶-Val¹¹) and p12.

25. The DNA vaccine according to claim 23 further comprises a CpG motif.

26. The DNA vaccine according to claim 24, wherein said CpG motif has a sequence comprising the dinucleotide CG flanked at the 5′ side by two purines and at the 3′ side by two pyrimidines.

27. The DNA vaccine according to claim 24, wherein said CpG motif comprises AACGTT.
